(19) United States
(12) Patent Application Publication
(10) Pub. No.: US 2009/0038932 A1
(43) Pub. Date: Feb. 12, 2009

(54) DEVICE AND METHOD FOR NONINVASIVE ULTRASONIC TREATMENT OF FLUIDS AND MATERIALS IN CONDUITS AND CYLINDRICAL CONTAINERS

(75) Inventors: Kayte M. Denslow, Richland, WA (US); Gerald J. Posakony, Richland, WA (US); Leonard J. Bond, Kennewick, WA (US); Aaron A. Diaz, Richland, WA (US); Mikhail S. Alnajjar, Richland, WA (US); James A. Franz, Kennewick, WA (US)

Correspondence Address: BATTELLE MEMORIAL INSTITUTE;ATTN: IP SERVICES, K1-53, P. O. BOX 999, RICHLAND, WA, 99352, US

(73) Assignee: Battelle Memorial Institute

(21) Appl. No.: 11/835,854

(22) Filed: Aug. 8, 2007

(51) Int. Cl. B01J 19/12; C07C 1/00

(52) U.S. Cl. 204/157.15; 204/193

(57) ABSTRACT

A system, method, and device are described for ultrasonic treatment of viscous fluids, including, e.g., crude oils that provide a variety of desired modifications. The invention includes a container having a circumvolving outer wall configured to allow passage of a quantity of a material within a passageway therein and ultrasonic transducers that attach to and circumvolve the outer wall of the container. The ultrasonic transducers transmit ultrasonic energy into material within the container at preselected frequencies thereby achieving desired effects.

### FIELD OF THE INVENTION

The invention relates generally to ultrasonic devices and methods for treatment of fluids, including, e.g., crude oils and other fluids in a pipe (e.g., a crude oil production pipe), a tube (e.g., microfluidic tubes), or cylindrical container (e.g., a chemical batch reactor). In preferred embodiments, the invention relates to devices and methods for introducing and utilizing non-cavitating and/or cavitating ultrasonic energy to provide a variety of desired physical and chemical property modifications to fluids and materials, including, e.g., cleavage of chemical bonds and formation of reactive radicals; changing viscosity, inducing rapid heating, and separating phases in multiphase systems.

### BACKGROUND OF THE INVENTION

Ultrasound is a pressure wave and a form of vibrational mechanical energy. When ultrasound propagates through, and interacts with, a fluid, the energy is attenuated by scattering or absorption. At lower sub-cavitational powers, ultrasonic energy is absorbed by the fluid in a thermal interaction that causes local viscoelastic heating. At higher cavitational powers, interactions become increasingly non-linear and both non-thermal mechanical interactions and cavitational mechanisms become significant. Non-thermal mechanical interactions can include radiative pressures, interaction forces, acoustic streaming, and hydrodynamic shear forces. Ultrasonic interactions with fluids, particularly those involving cavitation, have been exploited for many years with devices that perform a variety of functions.

One of the most prominent ultrasound devices utilized in these ultrasonic interactions includes sonic horns or probes. Ultrasound probes are typically inserted into a material of interest to transmit ultrasonic energy into the material. However, ultrasonic probes are prone to a variety of problems related to their use, including, but not limited to, e.g., degradation and/or erosion of the tips of the probes as a function of age (e.g., hours of use), subsequent contamination of materials into which these probes with tip probe particulates are placed, increasingly inefficient transmission of ultrasonic energy with increasing probe tip erosion, disruption of flow of materials around the probes, small focal volumes or areas of effectiveness, and other related problems. In batch ultrasound devices, e.g., cleaning devices, sonic energy is typically unfocused and is further limited for flow processing and applications.

Magnetostrictive (also called “magneto-restrictive”) systems known in the art rely on a double conversion: a first conversion from electrical energy to magnetic energy and a second conversion from magnetic energy to mechanical energy to produce a sound wave. Such systems are generally limited to frequencies in the 30-50 kHz range; and the magnetic systems are usually less than 50% efficient due to the energy lost in heating of coils and effects of magnetic hysteresis. Additionally, generators used in conjunction with these systems, even if well tuned, are generally no more than 70% efficient, meaning that overall power delivery is between 35% and 40% efficient. With increasing energy costs, the impact on operational costs of a large magnetostrictive system cannot be underestimated.

Accordingly, systems, devices, and processes are needed that provide increased efficacy and efficiency in transmission of ultrasonic energy into materials that overcome these and other related problems associated with prior art devices.

The objects, advantages, and novel features of the present invention will be set forth as follows and will be readily apparent from the descriptions and demonstrations set forth herein. Accordingly, following are descriptions of the present invention that should be seen as illustrative of the invention and not as limiting in any way.

### SUMMARY OF THE INVENTION

The present invention is a device and method for processing of fluids, including, e.g., crude oil and other viscous and light fluids, through use of an ultrasonic energy transmitting device that attaches to, and substantially surrounds, a conduit, a tube, or a container in which fluids, or other materials are placed. By substantially surrounding the conduit, tube, or container onto which the transmitting device is placed and transmitting focused and unfocused ultrasonic energy into the contained fluid or material in a way that does not require an intrusive foreign body to be placed into the fluid or material, various problems associated with prior art devices are overcome. Furthermore, as the detailed descriptions further clarify, such configurations provide exceptional advantages over the prior art in terms of greater efficacy and efficiency in transmitting energy to the material or fluid of interest and provides for a variety of treatment options and advantages over the prior art.

In descriptions of the preferred embodiment of the invention set forth hereafter, intense, cylindrically focused ultrasonic energy produced by the invention has been shown to: cleave chemical bonds and form reactive radicals; induce rapid heating in fluids, including, e.g., crude oils and other viscous Fluids; and separate emulsions using both cavitating and non-cavitating ultrasonic energy. In other embodiments, the invention has been utilized with other additives to enhance effectiveness of the various methodologies described herein. To encourage formation of favorable products from ultrasonic cavitation, use of water or other co-solvents and/or catalysts may be necessary, depending on chemical nature of the treated system. It is reasonable to believe that ultrasonic vibrational energy provided by the invention may also mitigate accumulation of pipe-fouling deposits on the inner diameters of pipe walls. The robustness, adaptability, noninvasiveness, and large sonication volume provided by the invention allows ultrasonic processing of fluids and materials to move beyond laboratory scale processing to industrial scale processing for a variety of fluid and/or material processing applications.

While the present invention will be discussed in conjunction with a preferred embodiment of the invention related to modification of crude oil, it is to be distinctly understood that the invention is not limited thereto. It is anticipated that the invention may be appropriately utilized in a variety of other industries, materials, devices, and applications. These include, but are not limited to, chemical applications; petrochemical applications; materials chemistry; industrial applications that achieve, e.g., enhanced chemical reaction rates, mixing, enhanced catalysis of reactions, preparation and activation of catalysts, bond breaking, formation of free radicals, polymerization and depolymerization of long-chain molecules, and the like; pharmaceutical applications such as drug synthesis, premixing, dispersion/suspension, cracking enteric coatings for dissolution testing, degassing and homogenation of slurries, and the like; medical applications such as performing extracorporeal shockwave lithotripsy, enhancing chemotherapeutic effects of drugs, destruction of cancerous growths, dissolution of blood clots, inclusion in advanced surgical techniques, and therapeutic ultrasonic methods, and the like; biological and biotechnological applications including such actions as cell lysis, release and extraction of enzymes and proteins from cells, cell disruption and enzyme purification and the like; environmental and radiochemical applications, including such tasks as degrading pollutants and volatile organic compounds, processing soil and sediment samples, enhancing actinide separations, as well as promoting dissolutions and oxidation processes; and microfluidic applications. As ultrasonic transmitting devices described herein are not limited by size, in other embodiments, the invention may be adapted and scaled for use in conjunction with microfluidic applications, instruments, and devices where, e.g., fluids and/or materials introduced to microchannel tubing and devices are treated with ultrasonic energy to bring about desired results and modifications as listed and/or described herein. Thus, no limitations are intended.

The purpose of the foregoing abstract is to enable the United States Patent and Trademark Office and the public generally, especially scientists, engineers, and practitioners in the art not familiar with patent or legal terms or phraseology, to determine quickly from a cursory inspection, the nature and essence of the technical disclosure of the application. The abstract is neither intended to define the invention of the application, which is measured by the claims, nor is it intended to be limiting as to the scope of the invention in any way.

Various advantages and novel features of the present invention are described herein. By way of illustration of the best mode contemplated for carrying out the invention, a preferred embodiment of the invention is shown and described. As will be readily apparent to those skilled in the art from the following detailed description, the invention is capable of modification in various respects without departing from the true spirit of the invention. Accordingly, the drawings and description of the preferred embodiment set forth hereafter are to be regarded as illustrative in nature, not restrictive.

### BRIEF DESCRIPTION OF THE DRAWINGS

FIG. 1 is a perspective view of a preferred embodiment of the invention.

FIG. 2 is an end view of the embodiment of the invention shown in FIG. 1.

FIGS. 3a-3b are perspective views illustrating different transducer configurations, according to two embodiments of the invention.

FIGS. 4a-4b are end views illustrating different transducer geometries, according to two embodiments of the invention.

FIG. 5 is a first perspective view of the preferred embodiment of the invention incorporated in a test configuration of a device for treatment of, e.g., crude oils.

FIG. 6 is a second perspective view of the preferred embodiment of the invention in a test configuration of a device for treatment of, e.g., crude oils.

FIG. 7 is a chart showing results of the preferred embodiment of the present invention in ultrasonically heating a crude oil.

FIG. 8 is a chart correlating features of the preferred embodiment of the invention with those of a conventional heating method known in the art.

### DETAILED DESCRIPTION OF THE PREFERRED EMBODIMENTS OF THE INVENTION

Following is a description of preferred and best modes for embodiments of the present invention. It will be clear from this description that the invention is not limited to the illustrated embodiments but also includes a variety of modifications, alternative constructions, and embodiments thereto. Thus, it should be understood that there is no intention to limit the invention to the specific forms disclosed. On the contrary, the invention covers all modifications, alternative constructions, and equivalents falling within the spirit and scope of the invention as defined by the claims. Therefore the description should be seen as illustrative and not limiting. Figures described hereafter refer to preferred embodiments of the present invention. While the preferred embodiments of the invention are configured and described herein for volumetric treatment of viscous fluids, particularly low-grade and heavy-grade crude oils, the invention is not limited thereto but may be variously embodied for use in other applications, which may include a variety of modifications, alterations, and constructions including additional components or pieces for obtaining a desired and designated result. Thus, no limitations are intended. The invention described herein is capable of operating at cavitational and sub-cavitational powers in fluids having a range of viscosities, and at multiple ultrasonic frequencies, to achieve, for example, cleavage of chemical bonds and formation of reactive radicals, viscosity changes, rapid heating, and separation of phases in multiphase systems.

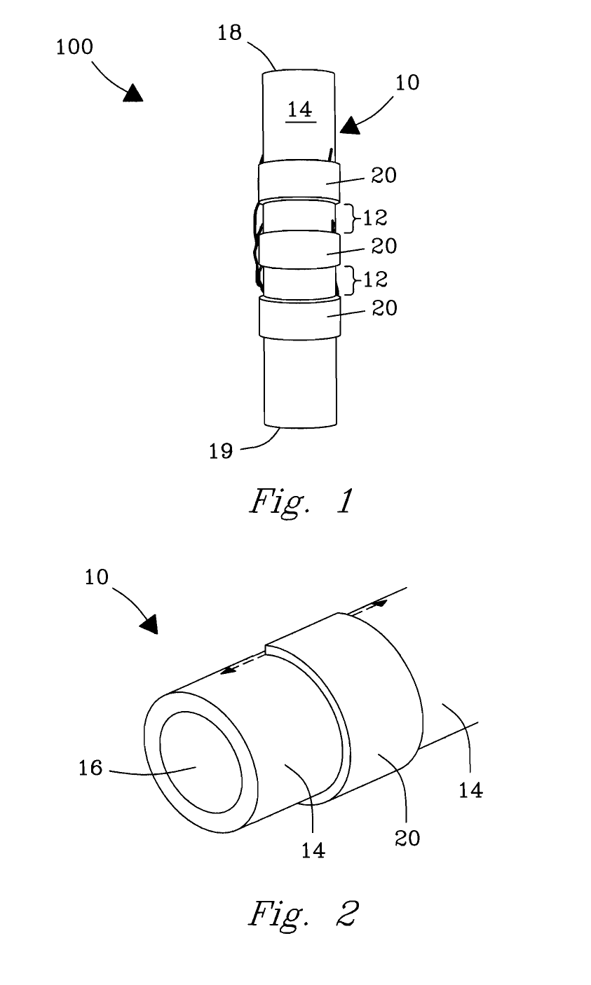

FIG. 1 shows device 100 according to an embodiment of the invention, made of a walled conduit or container 10 having an outer wall 14 that extends along a length of the conduit from a first end 18 to a second end 19. Outer wall 14 may be made of any material designated for the use in any particular application including, e.g., glass, metal, and plastics. In some cases, it is anticipated that no conduit may be required at all, and that a target material may be directly acted upon by the ultrasonic device of the present invention. At least one ultrasonic transmitting device 20 (transducer) is connected to the outside surface of outer wall 14 in such a way as to allow transmission of ultrasonic energy through the outerwall into a target material introduced to the conduit or container.

In a preferred embodiment of the invention, ultrasonic transmitting device 20 is a generally cylindrically shaped ultrasonic transducer ring made, e.g., of a lead zirconate titanate (PZT) piezoelectric ceramic (Channel Industries, Santa Barbara, Calif., USA) that is mounted to the outside of a cylindrical tube or is in direct contact with a fluid or material of interest. While in this preferred and presently described embodiment of the invention, conduit 10 is a section of pipe, it is to be distinctly understood that use of the term “walled container” includes conduits such as pipes and tubing as well as other types of containers such as those having closed tops or bottoms or both, e.g., a chemical batch reactor. Thus, no limitations are intended. It is also to be distinctly understood that the invention covers all forms and materials from which these items can be constructed.

In the instant embodiment, a plurality of transducer rings 20 are carefully mounted onto outer wall 14 of conduit 10 using a high affinity bonding method such as high temperature epoxy to ensure that no air bubbles exist between the rings and the conduit or container. Bonding material is selected that best matches acoustic impedances of the transducer rings and the conduit, tube, or container to provide efficient transmission of ultrasonic energy into the fluid or material of interest contained therein. In the instant embodiment, transducer rings are spaced at a separation distance 12 of at least about one-half the wavelength of the intended operating frequency of the transducer rings used for the conduit or container. Here, the minimum one-half wavelength spacing ensures overlap of sound fields emitted by each of the rings at the selected operating frequency, providing a concerted volumetric coverage and good acoustic resonance.

Spacing between transducers depends on intended operating frequencies. Thus no limitations in spacing are intended. For example, transducers may be placed closely together, with little to no spacing between adjacent transducers so as to provide significant overlap of acoustic energy. In other configurations, the ultrasonic transmitting device or transducer can include the conduit or container wall as a component of the transducer, where the wall acts as a resonant component of the transducer and/or matching layer. In various applications, spacing may be selected at intervals or multiples of, e.g., ¼-½ of a wavelength or greater, achieving reinforcement in the ultrasound energy delivered into the conduit or container. All wavelengths and frequencies as will be selected by those of skill in the art in view of the disclosure are within the scope of the invention. In short, spacing will be application dependent in order to achieve desired effects. Effects and modifications include, but are not limited to, e.g., 1) sonochemical effects (e.g., chemical bond cleavage and reactive radical formation), 2) physical property modifications (e.g., decreased viscosity, heating, or other physical property changes), 3) thermophysical effects, 4) viscoelastic property effects, 5) electrical properties effects (e.g., changes in resistance), and 6) fluid properties effects.

FIG. 2 illustrates an end view of the preferred embodiment of the invention described previously in reference to FIG. 1. Outer wall 14 of conduit 10 defines a passageway 16 into which a target material is positioned or introduced to be processed. In the figure, one transducer ring 20 is illustrated, but is not limited thereto. In one example application utilizing crude oil as a target material, outer wall 14 is made of steel. It is to be distinctly understood that the invention is not limited to processes and/or treatments involving crude oils nor to outer walls configured with steel, but may be otherwise variously configured to meet needs and necessities of a particular use or application.

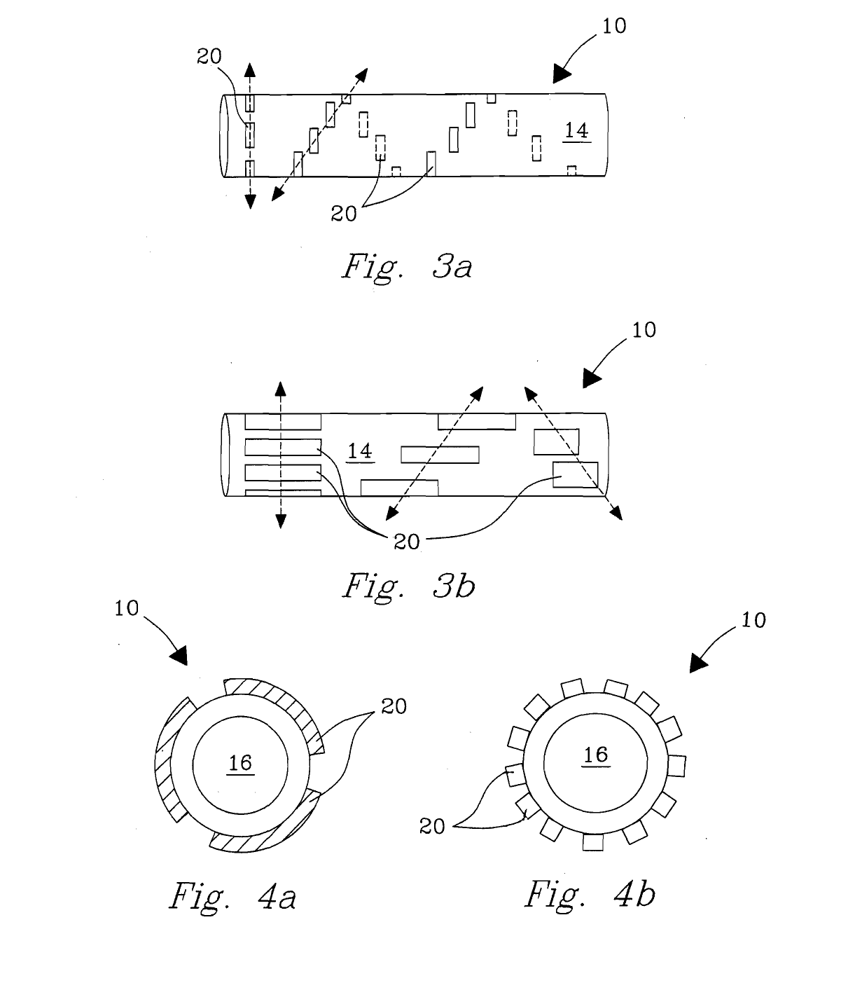

FIGS. 3a-3b present perspective views of various transducer configurations, according to different embodiments of the invention. In FIG. 3a, ultrasonic transmitting devices 20 (transducers) are segmented pieces of various lengths. The various pieces are attached to the outside of conduit or container 10 so as to circumvolve outer wall 14 around the passageway (FIG. 2). As illustrated, the transducers are vertically oriented, with the circumvolving pieces assembled, e.g., in a singular plane or in a helical pattern or in other alternative configurations. Configurations are not limited. In FIG. 3b, for example, transducer 20 segments (elements) again circumvolve outer wall 14 of conduit 10 and are horizontally oriented, but may be positioned in various and alternative configurations or arrangements about the conduit or container. Configurations include, but are not limited to, e.g., configurations involving transducer (element) strips of various length, spiral configurations, and stacked configurations with elements positioned in single or multiple planes. In addition, transducers may also be of various dimensions and thicknesses depending on intended applications and modes of operation. Thus, no limitations are intended. In other alternate configurations, transducer elements could also be arranged to circumvolve outer walls of multiple containers that are linked, e.g., in series for ultrasonic treatment of a fluid or material in a multi-stage or static multi-stage processing operation. In another alternate configuration, multiple transducer elements could be stacked in a static reactor configuration permitting operation in a single mode, or simultaneous operation in two or more modes, e.g., for sonochemical treatment of fluids or materials.

As will be understood by those of skill in the art, operating frequencies of transducers and transducer elements are selected based on desired effects, modifications, and/or properties to be achieved from ultrasonic treatment of the fluid or material of interest, and is therefore not limited. Typical operating frequencies can range, e.g., from low kilohertz frequencies (e.g., 10 kHz to 50 kHz) to low megahertz frequencies (e.g., 1 MHz to 5 MHz). Choice of frequency is also a function of the physical properties (e.g., viscosity) and ultrasonic attenuation of the fluid or material to be treated.

FIGS. 4a-4b illustrate two end views of a section of conduit 10 or container 10 presenting different exemplary transducer geometries, according to two different embodiments of the invention. In FIG. 4a, transducers 20 are segmented, of a uniform thickness, and circumvolve conduit 10 around the passageway 16. Another transducer geometry is illustrated in FIG. 4b. Here, transducers 20 are segmented, and circumvolve conduit 10 around the passageway 16 and are of a generally square or rectangular geometry. Other geometries may be equally workable for transmitting and focusing ultrasonic energy depending on the intended applications, processing conditions, and operating frequencies. All geometries as will be selected by those of skill in the art in view of the disclosure are within the scope of the invention. No limitations are intended.

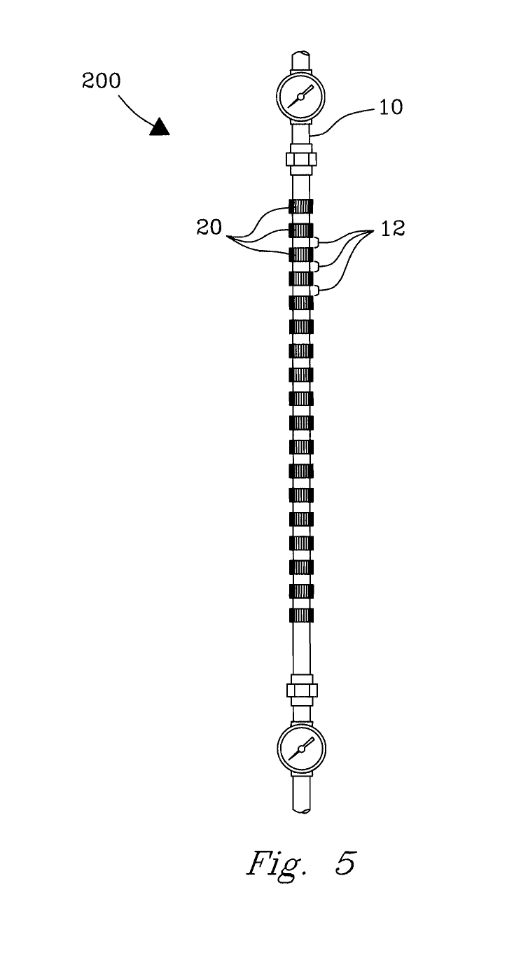

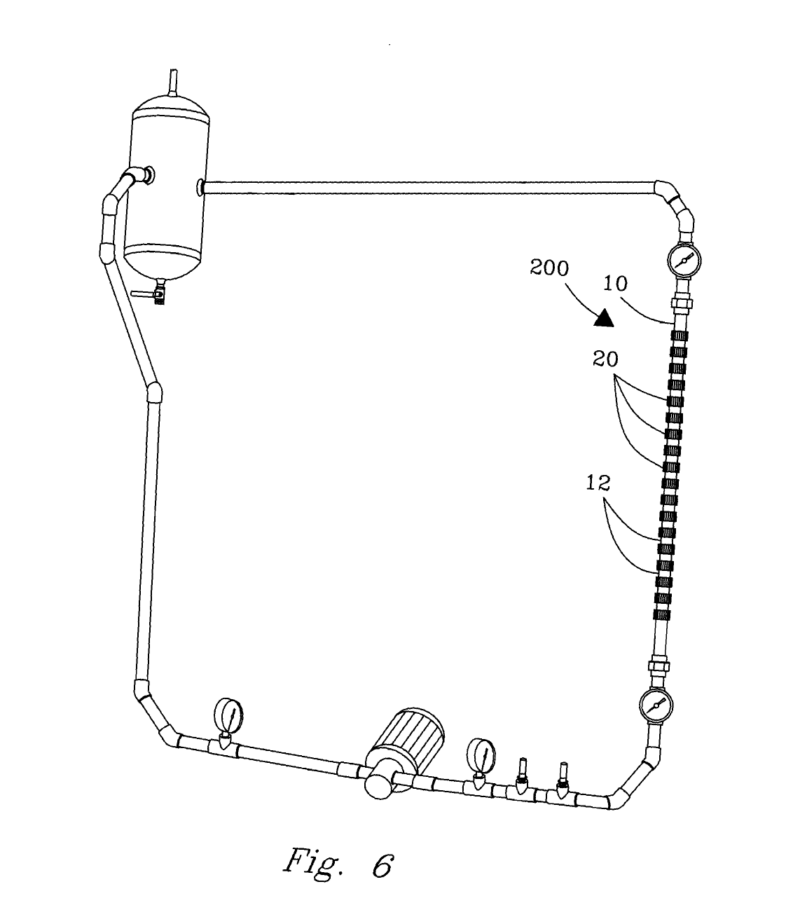

FIG. 5 is a perspective view of a preferred embodiment of the invention that illustrates a device 200 for treatment of crude oils, showing a vertically-oriented half-scale flow loop conduit 10 representative of a sub-sea or sub-terrain crude oil transport pipe. Flow loop 10 includes a plurality of ultrasound transmitting transducer rings 20 positioned such that separation distance 12 between each of the rings is at least about one-half wavelength of the selected operating frequency of the reactor transducer rings along the length of the conduit, as described previously in reference to FIG. 1, but is not limited thereto. FIG. 6 is another perspective view of the preferred embodiment of the invention incorporated in a test configuration of device 200 for treatment of crude oils, showing conduit 10, and transducer rings 20 positioned a separation distance 12, as previously illustrated in FIG. 5.

In processing trials conducted on crude oils in both static and dynamic flow conditions, the ultrasonic transmitting rings were made of a lead zirconate titanate (PZT) piezoelectric ceramic material. PZT was chosen for use given its high coupling factors and high piezoelectric and dielectric constants over extended temperature ranges and stress amplitudes. PZT-8 (Channel Industries, Santa Barbara, Calif., USA) was a preferred PZT material, chosen for its superior tensile strength, high mechanical “Q” value, high Curie point, and ability to handle high voltages. The PZT-8 delivered a nominal power of ≧6 W/cm² in applications for which it was employed, but is not limited thereto. For example, acoustic power within the cylindrical configuration of the preferred embodiment of the invention can exceed 100 W/cm². And, it should be strictly understood that while PZT ceramics are described herein in conjunction with this embodiment of the invention, transducer materials are not limited. Choice of transducer materials depends on desired physical parameters, including, e.g., acoustic properties, frequencies and modalities, power generation, and the like. All transducer materials as will be selected by those of skill in the art in view of the disclosure are within the scope of the invention.

For the ultrasonic fluid processing described herein, a ring geometry was chosen for its ability to focus ultrasonic energy into the center axial zone of a cylindrical conduit or container. In the preferred embodiment of the invention, the PZT rings of the prototype reactors had geometries and dimensions that varied, thus providing different resonance frequencies. PZT material described herein is primarily voltage driven. Efficiency of the PZT rings and properties of fluids and materials being sonicated determine the maximum electrical power the rings respond to. In general, the greater the frequency employed in application processing, the greater the heating capacity for treatment of fluids.

Piezoelectric ceramic transducers are extremely efficient due to their ability to directly convert electrical energy to mechanical energy in a single step. Direct application of power to a piezoelectrically active ceramic causes it to change shape and create the sound wave. And, energy losses due to internal friction and heat in these ceramics are typically less than 5%. Thus, up to 95% of power delivered to the transducer is used to do sonication. Modern ultrasonic generators used to drive piezoelectric transducers are generally over 75% efficient making the overall system efficiency 70% or higher.

As will be understood by those of skill in the sonic arts, resonance frequencies and modes for specific transducer materials can be determined and/or selected based on standard dimension and piezoelectric coefficients as will be provided, e.g., by commercial vendors or that are available in the published research literature.

Power generators and circuit networks can also be used to generate desired frequencies for desired applications and processing, including, e.g., non-resonance frequencies, resonance frequencies, and/or combinations of non-resonance/resonance frequencies. In addition, function generators can be used to generate various desired waveforms, including, e.g., continuous and pulsed waveforms. Thus, it should be understood that no limitations to specific resonance frequencies, waveforms, and physical parameters described herein are intended.

Other factors that influence resonance frequencies include the reactor or conduit wall thickness, materials to be processed within the reactor, power levels employed during processing, as well as media (e.g., air, water, soil, etc.) that are external to, or otherwise cover, conduits and conduit passageways.

In the cylindrical (ring) configuration of the preferred embodiment of the invention, the piezoelectric transducers permit both single mode and multimode operation. Frequency modes include, but are not limited to, e.g., length mode, thickness mode, hoop mode, radial mode, etc. In multimode operation, when, e.g., two or more modes are operated, e.g., in a stack configuration, the modes can be working simultaneously at the same or different frequencies.

In a preferred embodiment, the transducer rings possess three optimum resonance modes, which were all explored: 1) hoop mode, that transmits sonic energy in the diameter dimension along the transducer or transducer segment; 2) length mode, that transmits sonic energy in the length dimension along the transducer or transducer segment; and 3) thickness mode that transmits sonic energy in the wall thickness dimension in and out of the plane of the transducer or transducer segment. Hoop mode had the lowest operating frequency. Length mode was an intermediate frequency. Thickness mode had the highest operating frequency. At these preferred resonance frequencies, the PZT rings convert electrical energy into mechanical energy most efficiently. In addition, harmonics and sub-harmonics of these same resonance frequencies may also be effective. Two or all three of the modes of the PZT rings can be used simultaneously if the rings have appropriate dimensions. If all three modes of the rings are to be employed in succession (i.e., one at a time), spacing between the rings would be based on the highest operating frequency (shortest wavelength).

In general, power efficiency is increased if electrical impedances of the driving electronics are matched to the electrical impedance of the PZT transducer load, thereby achieving matching resonance frequencies. A resonance frequency of the PZT rings lies between two frequencies: 1) the “minimum impedance frequency” and 2) the “maximum impedance frequency”. Although the frequencies of the minimum and maximum impedance frequencies are close to each other, they have very different electrical impedance values. The minimum impedance frequency has a very low electrical impedance value; the maximum impedance frequency has a higher electrical impedance value. Impedance is different at each resonance frequency of the PZT rings, with an overall trend in impedance that decreases with increasing resonance frequency. Further discussion of electrical impedance matching is presented in Example 1.

### Ultrasonic Cavitation of Fluids

The preferred embodiment of the present invention is capable of achieving high power to induce ultrasonic cavitation. Cavitation has been demonstrated to break chemical bonds, generate useful chemical radicals, break up contaminants, and affect other associated sonochemical phenomena. Cavitation requires ‘pumping’ ultrasonic energy at a power level and an associated frequency that produces bubble collapse within a fluid or material. “Cavitation threshold”, or the point at which cavitation begins, is a function of temperature, particulates in a fluid or material to be treated, and other non-linear interactions. Bubble collapse associated with cavitation produces high temperatures and pressures that can cause changes in the molecular structure of a fluid, a slurry (particles in suspension), or a compound. This phenomenon enhances, e.g., chemical reaction rates and hydrocracking due to the formation of radicals and cleavage of bonds. In general, cavitation threshold for fluids increases over about 4 orders of magnitude in the frequency domain. Acoustic frequencies from the audible range into the ultrasonic range (400 Hz to 1 MHz and higher) are employed in modifying chemical reactions to generate greater yields with a corresponding increase in rates of the reactions under investigation. As intensity of acoustic energy in a fluid medium is increased, a point is reached at which intermolecular forces are not able to hold a molecular structure intact. Consequently, a molecular structure breaks down and cavitation bubbles are created. These bubbles expand and subsequently implode, giving rise to temperatures and pressures that can reach over 5000 K and several hundred atmospheres. Although the lifetime of an individual bubble may be a fraction of a second and the energy released by a bubble may be minimal, the cumulative energy involved in reactions is significant. For an individual bubble-cavitation cycle, energy can be concentrated by 12-13 orders of magnitude. This concentrated energy enhances reaction rates because of the cleavage of chemical bonds and subsequent formation of reactive radicals. These cavitation-induced effects can cause physical, chemical, and biological effects in a fluid. Successful cleavage of chemical bonds using ultrasound technology has been demonstrated, and is described further in Examples hereafter.

### Treatment of Emulsions

In nature, crude oils are typically co-mingled with water and gases as emulsions. The term “emulsion” as used herein refers to any of the plurality of mixtures comprised of oils, gases, and water. Such mixtures exist in various forms, e.g., as mixtures of oil droplets in water or gases; mixtures containing separated or distinct phases; and mixtures containing multiple phases, e.g., two or three phase systems. Oil field emulsions can be classified into three broad groups: 1) water-in-oil emulsions (most common in the oil industry), 2) oil-in-water emulsions, and 3) multiple or complex emulsions. Emulsions create a number of problems and can be encountered in almost all phases of oil production and processing, e.g., inside reservoirs, well bores, well heads, wet crude-handling facilities, transportation through pipelines, and crude storage during petroleum processing. Emulsions are difficult to treat and cause a number of operational problems such as tripping of separation equipment in gas/oil separating plants, production of off-specification crude oils, and creating high pressure drops in flow lines. In addition, emulsions must be treated to remove dispersed water and associated inorganic salts in order to meet crude specifications for transportation, storage, export, as well as to reduce corrosion and catalyst poisoning in downstream-processing facilities. Emulsions are stabilized by emulsifiers that tend to concentrate at the oil/water interface where they form interfacial films. This generally leads to a reduction of interfacial tension and promotes dispersion and emulsification of the droplets. Naturally occurring emulsifiers in the crude oil include higher boiling-point fractions, such as asphaltenes, resins, and organic acids and bases. These compounds are believed to be main constituents of interfacial films, which form around water droplets in an oil field emulsion. Interfacial films are believed to result from the adsorption of high-molecular weight polar molecules that are interfacially active (i.e. exhibit surfactant-like behavior) and enhance the stability of emulsions. Demulsification is the process by which these crude oil emulsions are disrupted and droplets made to coalesce into separate oil and water phases. Successful treatment of a 38 wt % brine-in-crude oil emulsion has been demonstrated using the preferred embodiment of the present invention, described in Examples hereafter.

### EXAMPLE 1

### (Exemplary Operating and Processing Conditions)

In an exemplary, non-limiting processing operation, the PZT rings were powered by a commercially available 50-ohm arbitrary output waveform function generator (Hewlett Packard, Palo Alto, Calif., USA) and a commercially available 50-ohm output radio frequency (RF) power amplifier (Electronics & Innovation, Rochester, N.Y., USA). The function generator was tuned to provide a continuous or pulsed sinusoidal wave of a predetermined frequency and voltage. Output signal from the function generator was provided to the input of the RF power amplifier where it was amplified before being supplied to the PZT rings. The PZT rings were wired in parallel to deliver a uniform voltage.

A perpetual mismatch was observed between the 50-ohm electronics and the inherent electrical impedances of the PZT rings at their resonance frequencies (i.e., the load). This impedance mismatch affected overall efficiency of the system, requiring the amplifier to generate more power than would be necessary with matching impedances. The impedance mismatch also limits the performance of the PZT rings. For example, PZT rings used in testing demonstrations described here are capable of handling 300 V/mm. However, this energy capacity could not be reached due to power that was reflected back to the amplifier as a consequence of the impedance mismatch between the amplifier and the PZT load. A threshold exists in the power reflected to the RF power amplifier that when exceeded engages the amplifier's overload safety switch that shuts the amplifier off. An impedance matching network (Sonic Concepts, Bothel, Wash.) can be used to match impedances in some instances. In the instant case, an impedance matching network was not deemed an effective solution due to the large difference between the 50-ohm driving electronics and the low electrical impedances of the PZT rings at their resonance frequencies. Here customized driving electronics were an effective solution that would have either an output impedance matched with that of one PZT resonance frequency or have a variable output impedance that would accommodate all the impedances of the PZT rings at all of their resonance frequencies. Alternatives to building customized electronics would be to 1) find PZT rings that naturally have impedance values close to 50-ohms at a desired resonance frequency or 2) connect a sufficient quantity of PZT rings in parallel to reach an impedance value close to 50-ohms without compromising the power that each PZT ring needs for optimal performance, as amperage does drop with each PZT wired in parallel. Customized electronics can be used to achieve desired operating parameters and/or to improve PZT ring performance. Choice of electronics may also improve overall system efficiency, by, e.g., decreasing power that may be reflected back to the driving electronics due to impedance mismatches, thereby achieving lower energy conversion to heat in the driving electronics.

While a 50-ohm generator and power amplifier have been described here in conjunction with the present embodiment, components are exemplary and the embodiment is not limited thereto. Choices for components and equipment will depend on intended applications, as will be understood by those of skill in the art. Thus, no limitations are intended.

As described previously herein, each operating frequency of the PZT rings has a different electrical impedance that is less or greater than the output impedance of the RF power amplifier. The impedance mismatch causes a significant portion of the power the amplifier attempts to deliver to the load (the PZT rings) to be reflected back to the amplifier as heat. If the reflected power exceeds a threshold, the amplifier shuts down. Reflected power is affected by input voltage from the function generator and the impedance of the load. At each operating frequency, the PZT rings (the load) was provided with the maximum power the amplifier could deliver without exceeding the reflected power threshold of the amplifier. The larger the mismatch in impedance between the amplifier and the load, the smaller the input voltage could be and, thus, the less power that could be delivered to the load. And, since the impedance of the load was different at each frequency, the power delivered to the load was different for each frequency. Depending on the frequency, the input voltages delivered to the PZT rings ranged from about 100 Vₚₚ to about 430 Vₚₚ. Placing an impedance matching network in-line with driving electronics and PZT load, or building customized driving electronics with output impedances that match PZT load, are expected to increase power delivery to the PZT load and improve system efficiency.

### EXAMPLE 2

### (Static Mode Processing of Mexican Crude Oil)

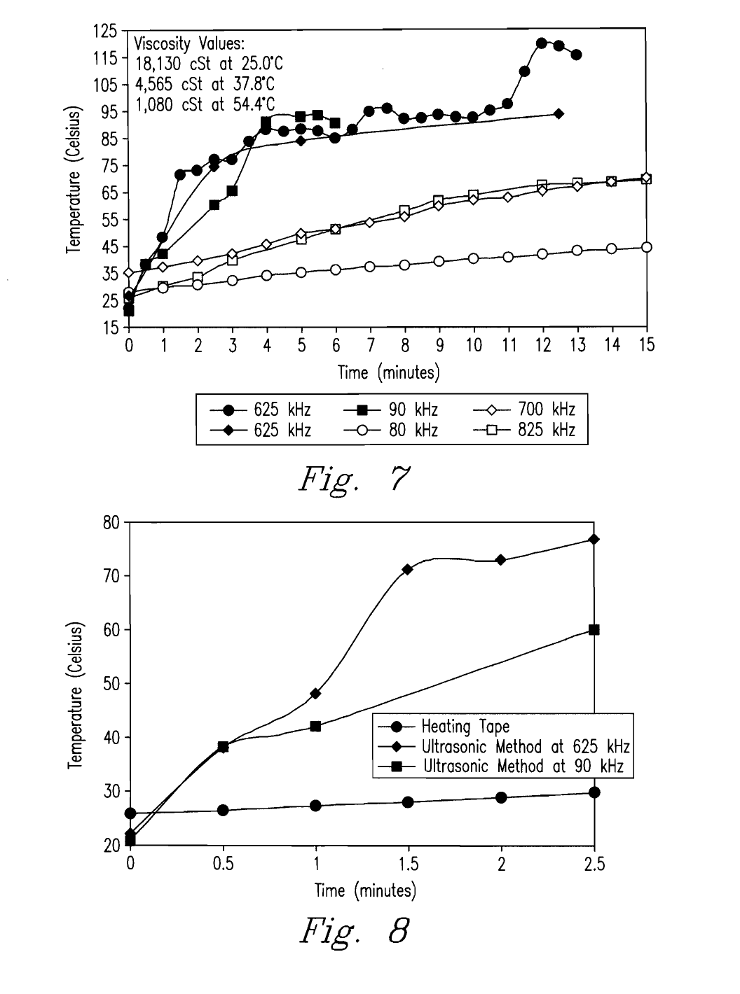

A low grade, heavy (i.e., 12.67 API) Mexican crude oil was processed in static mode 1) at resonance frequencies of 625 kHz (thickness mode) and 90 kHz (length mode) and 2) off-resonance frequencies of 80 kHz, 700 kHz, and 825 kHz. In the preferred embodiment of the invention configured for static processing of crude oils, container 10 had a wall thickness of 3.264 mm and a length of 20.71 mm. PZT rings 20 had dimensions of: 62.23 mm O.D.; 55.63 mm I.D. Outerwall 14 was constructed of a 64 mm O.D. Schedule-40 carbon steel pipe segment, chosen due to its use in crude oil production piping. Three (3) PZT rings were mounted on the outside of the pipe segment. Temperature data during static sonication of the crude oil are presented in Tables 1 and 2. Viscosity data are presented in Table 2. FIG. 7 plots changes in temperature of the crude oil in the center axial zone of the reactor as a function of sonication time.

TABLE 1
Temperature Profile during Static Sonication of Heavy (12.67 API)
Mexican Crude Oil.

| | | |
|---|---|---|
| Time (minutes) | Time (seconds) | Temperature (Celsius) |

625 kHz Thickness Mode, Resonance Frequency (solid circle)

| | | |
|---|---|---|
| 0 | 0 | 22.1 |
| 0.5 | 30 | 38.1 |
| 1 | 60 | 48.1 |
| 1.5 | 90 | 71.2 |
| 2 | 120 | 73 |
| 2.5 | 150 | 76.8 |
| 3 | 180 | 76.9 |
| 3.5 | 210 | 83.6 |
| 4 | 240 | 88 |
| 4.5 | 270 | 87.4 |
| 5 | 300 | 88.3 |
| 5.5 | 330 | 87.4 |
| 6 | 360 | 84.7 |
| 6.5 | 390 | 87.9 |
| 7 | 420 | 94.5 |
| 7.5 | 450 | 95.5 |
| 8 | 480 | 92 |
| 8.5 | 510 | 92.1 |
| 9 | 540 | 93.2 |
| 9.5 | 570 | 92.4 |
| 10 | 600 | 92.1 |
| 10.5 | 630 | 94.7 |
| 11 | 660 | 97.1 |
| 11.5 | 690 | 109.1 |
| 12 | 720 | 119.2 |
| 12.5 | 750 | 118.5 |
| 13 | 780 | 115 |

625 kHz Thickness Mode, Resonance Frequency (solid diamond)

| | | |
|---|---|---|
| 0 | 0 | 25.6 |
| 2.5 | 150 | 74.3 |
| 5 | 300 | 83.8 |
| 12.5 | 750 | 93.3 |

90 kHz Length Mode, Resonance Frequency (solid square)

| | | |
|---|---|---|
| 0 | 0 | 20.7 |
| 0.5 | 30 | 38.2 |
| 1 | 60 | 42.1 |
| 2.5 | 150 | 60 |
| 3 | 180 | 65.3 |
| 4 | 240 | 90.9 |
| 5 | 300 | 92.7 |
| 5.5 | 330 | 93.3 |
| 6 | 360 | 90.4 |

80 kHz Off-Resonance Frequency (hollow circle)

| | | |
|---|---|---|
| 0 | 0 | 28 |
| 1 | 60 | 29 |
| 2 | 120 | 31 |
| 3 | 180 | 32 |
| 4 | 240 | 34 |
| 5 | 300 | 35 |
| 6 | 360 | 36 |
| 7 | 420 | 37 |
| 8 | 480 | 37.8 |
| 9 | 540 | 38.9 |
| 10 | 600 | 40.0 |
| 11 | 660 | 40.6 |
| 12 | 720 | 41.7 |
| 13 | 780 | 42.8 |

700 kHz Off-Resonance Frequency (hollow diamond)

| | | |
|---|---|---|
| 0 | 0 | 35 |
| 1 | 60 | 37.2 |
| 2 | 120 | 39.4 |
| 3 | 180 | 42.2 |
| 4 | 240 | 45.6 |
| 5 | 300 | 49.4 |
| 6 | 360 | 51.1 |
| 7 | 420 | 53.3 |
| 8 | 480 | 55.6 |
| 9 | 540 | 59.4 |
| 10 | 600 | 61.7 |
| 11 | 660 | 62.8 |
| 12 | 720 | 65.0 |
| 13 | 780 | 66.7 |

825 kHz Off-Resonance Frequency (hollow square)

| | | |
|---|---|---|
| 0 | 0 | 26 |
| 1 | 60 | 30 |
| 2 | 120 | 33 |
| 3 | 180 | 39 |
| 5 | 300 | 47 |
| 6 | 360 | 51 |
| 8 | 480 | 57.8 |
| 9 | 540 | 61.7 |
| 10 | 600 | 63.3 |
| 12 | 720 | 67.2 |
| 13 | 780 | 67.8 |

TABLE 2
Temperature Profile and Viscosity during Static Sonication of Heavy
(12.67 API) Mexican Crude Oil.

| | Viscosity | |
|---|---|---|
| Oil Temperature (Celsius) | (centistokes) | Viscosity (m²/second) |
| 25.0 | 18,130 | 0.018130 |
| 37.8 | 4,565 | 0.004565 |
| 54.4 | 1,080 | 0.001080 |

Results in Table 1 and FIG. 7 show temperature increases monotonically with time until a steady or equilibrium state is reached. At a thickness mode frequency of 625 kHz, an input voltage of 150 Vₚₚ was required to achieve a 0.38° C./second rate of increase in temperature in the first 150 seconds. At a length mode frequency of 90 kHz, 430 Vₚₚ was required to achieve a 0.24° C./second rate of increase in temperature in the first 150 seconds. After 150 seconds, change in temperature with time was no longer linear as the viscosity of the crude oil and the viscoelastic heating decreased. More power could be applied to the PZT rings at these frequencies, especially 90 kHz. However, the maximum reflected power was reached at these respective input voltages. Data show that ultrasonic treatment of the invention is effective at rapidly heating the crude oil. Data in Table 2 further show that the technology is also effective in decreasing viscosity of the crude oil.

Rapid heating induced by the preferred embodiment of the invention described herein is a result of absorption of energy in the crude oil in response to ultrasonic wave propagation. As cylindrically focused ultrasonic waves travel through the viscous fluid, the fluid tends to oppose particle distribution motion of the ultrasonic waves, resulting in absorption of energy and conversion of the energy to heat. The more viscous the fluid, the greater the absorption of ultrasonic energy and the faster the rate of temperature increase. This absorption is not uniform in the fluid, however. Intensity of the incident wave is highest in the focal zone; absorption is also greatest in the focal zone. The wave energy that is absorbed irreversibly manifests itself in the form of heat, thus raising the temperature of the oil. It should be noted that the temperature would also be highest in the center axial zone of the cylindrical volume had the oil been subjected to planar waves because, under the influence of a distributed heat source in a body, thermal conduction processes always cause centerline temperature to be greatest. The situation is more complex here because uneven temperature distribution within the body of the oil sets up convection currents that alter the temperature profile. When temperature of the oil rises due to the application of the ultrasonic energy, the oil viscosity decreases. With a reduction of viscosity, the quantity of ultrasonic energy absorbed decreases and a lesser amount is converted to heat. Increase in temperature continues until thermal equilibrium is reached.

Various resonance frequencies and associated harmonic resonance frequencies were explored with the PZT rings. The resonance thickness mode and length mode provided superior results. Although 625 kHz (thickness mode) provided the most rapid heating at a lower voltage, 90 kHz (length mode) was selected as the most practical mode, as it provides a larger sonication volume (region). In particular, focal zones of the sound field are approximately 38 mm above and 38 mm below the ring, with activity in the entire sonication volume (region). For purposes of comparison, prior art horns with tips of ˜12.7 mm exhibit significantly small effective volumes, on the order of a few milliliters. In contrast, embodiments of the present invention provide sonification volumes on the order of hundreds of milliliters. Maximum achievable sonication volume with the PZT rings of the invention is dictated by size of the PZT ring, the frequency and power of operation, and the physical properties of the fluid system. “Sonoluminescence”, a term describing emission of light achieved by ultrasonic cavitation, is a direct indicator of cavitation. Sonoluminescense in water was used to qualitatively compare cavitation volumes provided by the present invention as compared to conventional ultrasonic horns. Cavitation volume provided by a single PZT ring described previously herein was estimated to be approximately 100 times greater than cavitation volume provided by a single-element probe of a commercial ultrasonic horn.

In thickness mode at 625 kHz, sonication is primarily achieved in the volume at the center of the ring. In hoop mode at 20 kHz, sonication is primarily achieved in the volume at the center of the ring and above and below the ring, but is not focused. In length mode at 90 kHz, sonication is primarily achieved in the volume at the center of the ring and above and below the ring, and is focused above and below the ring and occasionally along the entire longitudinal axis. Length mode was the preferred mode of operation when operating at a single frequency based on the volume and intensity of sonication.

Off-resonance frequencies of the PZT rings were also tested. At these off-resonance frequencies, the PZT rings do not efficiently convert electrical energy into mechanical energy, which results in sub-standard cavitations, heating, viscosity reduction, and mixing. Frequencies reported here are subject to change based on temperature changes the PZT rings experience during operation. During operation, driving frequency on the function generator is adjusted to compensate for changing frequency as the temperature of the reactor or pipe increases. When the overall system reaches thermal equilibrium, the resonance frequency stabilizes and adjustment of the driving frequency is no longer necessary. Results further demonstrated that rapid heating, viscosity reduction, and mixing of crude oil was achieved.

### EXAMPLE 3

### (Dynamic Flow Processing of Alaskan Crude Oil)

A heavy Alaskan crude oil (19 API), characterized as a 38 wt % brine-in-crude oil emulsion, was sonicated in the flow loop of the test configuration that contains the adapted crude oil processing technology (herein frequently referred to as the “test leg”) under dynamic flow processing conditions. The flow loop 10 was constructed of a 49.8 mm O.D. Schedule-40 carbon steel pipe. The flow loop comprised eighteen (18) PZT rings 20, but is not limited thereto. In the instant configuration, the PZT rings used for the flow loop were of a slightly different dimension than those used for static processing described in Example 2, including a 61.0 mm O.D.; 48.8 mm I.D.; 6.096 mm wall; and 30.48 mm length. Rings of the test leg were spaced 25.4 mm to 27.9 mm apart (i.e., at least 1.5 wavelengths at 65 kHz) to ensure constructive overlap of the sonicated volumes produced by neighboring rings. A 114 L (30 gallon) sample of the low grade, heavy (i.e., 19 API) Alaskan brine-in-crude oil emulsion filled the flow loop. The test leg sonicated the 19 API brine-in-crude oil emulsion as it was pumped through the flow loop at flow rates between about 340.7 mL/second to about 517.3 mL/second at 2.76 MPa. These flow rates and this pressure simulate conditions found in typical sub-terrain or sub-sea transport pipelines. Temperature of the oil before it entered the test leg and after it exited the test leg (delta T) was monitored with thermowell-encased, in-line thermocouples inserted into the cross-sectional center of the test leg at the oil entrance (T1) and exit points (T2). Delta T values of up to 0.45° C./second were achieved over a given sonication length, which depended on the number of rings energized and the spacing of the rings on the test leg. Although the test leg itself was 1.5 m long overall, the length of physical coverage on the O.D. of the test leg by the eighteen (18) rings was only 0.98 m. Depending on the number of rings energized and their spacing, the physical coverage of eight to twelve (8-12) rings on the O.D. of the test leg ranged from about 0.49 m to about 0.88 m.

Transducer ring operation in length mode (65 kHz) provided the most rapid heating and viscosity reduction. The most effective heating, provided a delta T value of 1° C. between the entrance and exit point of the flow loop, and included a 12 ring configuration covering a 0.7 m length of the test leg. This is a substantial temperature increase considering the crude oil flow rate and the short pipe length over which the temperature increase occurred. The rapid heating induced by the preferred embodiment of the invention described herein is a result of absorption of energy in the crude oil in response to ultrasonic wave propagation, similar to that described in Example 2.

In length mode, the PZT rings of the flow loop test leg exhibited a lower frequency than those described previously in reference to FIG. 1 since the rings on the flow loop test leg were longer. In the test configuration, other available operational modes of the PZT rings were: thickness mode, operated, e.g., at a frequency of 346 kHz; and hoop mode, operated, e.g., at a frequency of 19 kHz, which also demonstrated heating and mixing. Selection of the length mode frequency (65 Hz) was based on static batch testing described previously in Example 2, which was shown to provide mixing and the most rapid heating. Ring operation in the length mode offers a larger sonication volume than that provided in thickness mode. In length mode, the rings sonicate a relatively large volume of oil—38 mm below the center of each ring to 38 mm above the center of each ring—which translates to a volume of about 143 cm³ for each ring on the test leg. The other available resonance frequency was 19 kHz (hoop mode). This lower frequency demonstrated moderate heating and superior mixing, although operation at 19 kHz at high power is a noise nuisance.

The preferred embodiment of the invention demonstrated the ability to separate the 19 API 38 wt % brine-in-crude oil emulsion at a much lower temperature than that needed to break the emulsion with heat alone. A result of processing the emulsion in the flow loop was the separation of the emulsion (demulsification), which was realized at the conclusion of the dynamic testing after the flow loop was emptied of its 114 L sample. Approximately one-third (⅓) of the fluid emptied from the flow loop was a clear, white fluid, which is descriptive of brine. The maximum crude oil temperature reached during the dynamic testing was 54.9° C., a much lower temperature than the 111° C. temperature required to separate the emulsion with heat alone. Separation of the 38 wt % brine-in-crude oil emulsion was attributed all or in part to sonication.

### EXAMPLE 4

### (External vs Internal Heating)

Ultrasonic energy produced by the PZT rings is focused into the center of the fluid contained in the pipe or cylindrical container, and minimal energy is released to the environment around the PZT rings. The overall system is heated from the inside out. The focused ultrasonic energy causes the center of the oil in the pipe to heat first, then the surrounding oil, and finally the pipe and PZT rings. This was apparent when thermometer readings at the center of the test pipe or container would climb quickly, but the pipe itself remained cool to the touch. Air surrounding the outer diameters of the PZT rings has high acoustic impedance, and thus little acoustic energy is released to the air surrounding the PZT rings. Thus, energy is forced to travel through the pipe and crude oil. External heating methods (e.g., resistance heating, electrical trace heating, and the like), in contrast, lose a significant amount of energy to the surrounding environment and are not selective in what they heat. Heating tape, currently used to heat pipes, is designed for slow heating for temperature maintenance, not heat-up. A significant portion of heat provided by heating tape is spent first heating the pipe, pipe support, and valves, even when significant thermal insulation is present. Table 3 lists heating data resulting from static sonication of the heavy Mexican crude oil. FIG. 8 compares results obtained from static sonication of the heavy Mexican crude oil using ultrasonic technology of the present invention and an external heating method (e.g., heating tape, a form of electrical trace heating).

TABLE 3
Temperature Profiles from Static Sonication and External Heating of
Heavy (12.67 API) Mexican Crude Oil.

| | | |
|---|---|---|
| Time (minutes) | Time (seconds) | Temperature (Celsius) |

625 kHz Thickness Mode, Resonance Frequency (solid diamond)

| | | |
|---|---|---|
| 0 | 0 | 22.1 |
| 0.5 | 30 | 38.1 |
| 1 | 60 | 48.1 |
| 1.5 | 90 | 71.2 |
| 2 | 120 | 73 |
| 2.5 | 150 | 76.8 |

90 kHz Length Mode, Resonance Frequency (solid square)

| | | |
|---|---|---|
| 0 | 0 | 20.7 |
| 0.5 | 30 | 38.2 |
| 1 | 60 | 42.1 |
| 2.5 | 150 | 60 |

Heating Tape (solid circle)

| | | |
|---|---|---|
| 0 | 0 | 25.8 |
| 0.5 | 30 | 26.4 |
| 1 | 60 | 27.3 |
| 1.5 | 90 | 28 |
| 2 | 120 | 28.9 |
| 2.5 | 150 | 29.8 |

Results show the ultrasonic processing technology heated the crude oil much faster than heating tape, roughly 0.24° C./second to 0.38° C./second versus 0.027° C./second in the first 150 seconds of operation. Rapid rise in temperature at 625 kHz may be affected by any of the following physical factors in the reactor: 1) changes in flow, 2) changes in density, 3) localized changes in heating, 4) viscoelastic changes (e.g., damping), 5) changes in power, 6) changes in temperature, and 7) changes in chemical interactions, or other factors.

### EXAMPLE 5

### (Ultrasonic Cleavage of Chemical Bonds)

To demonstrate ability of the preferred embodiment of the present invention to cleave chemical bonds, model chemical markers were sonicated in prototype reactors. Markers included benzylphenylsulfide, toluene (methylbenzene), ethylbenzene, and isobutyl benzene. These chemical markers have chemical bonds representative of those found in sulfur-containing compounds found in crude oil. Model markers were selected given the ease of monitoring products formed from the bond cleavage. Successful cleavage of a C—H bond and removal of a hydrogen from the methyl side chain of a toluene molecule results in formation of a phenylmethyl radical. Two of these reactive radicals subsequently come together to form, among other products, bibenzyl. Production of bibenzyl was monitored as a measure of the success of sonication in achieving bond cleavage. Results from various trials are presented in Table 4.

TABLE 4
Product Results from Ultrasonic Treatment of Chemical Markers

| SUBSTRATE | PRODUCT (Bibenzyl)* |
|---|---|
| Toluene | 1 |
| Toluene/H₂O | 1.7 |
| Toluene/H₂O/H₂O₂ | 4 |

*Normalized concentrations, relative to bibenzyl formation, from sonication of pure toluene.

As shown in Table 4, the most successful bibenzyl results were obtained from tests involving sonication of pure toluene, and toluene that included cavitation-inducing additives such as water and hydrogen peroxide.

The present invention provides a significant advantage over other methods in the prior art and provides these advantages in an efficient manner from both an energy and cost perspective. The present invention provides for noninvasive, focused, high intensity ultrasonic processing of relatively large volumes of material in conjunction with ultrasonic devices of the invention that are operable at three different and optimum modes. These devices may be further utilized at different sub-harmonic and ultra-harmonic frequencies. The present invention allows for multiple modes (e.g., hoop and length modes) to be used simultaneously (despite different piezoelectric frequency constants for each mode) by cutting the PZT rings so that selected dimensions (e.g., mean diameter and length) are acoustically the same. These features provide the present invention with various abilities including: ability to focus ultrasonic energy into a cylindrical tube (e.g. pipe) and heat the contents, e.g., at the center axis of a pipe, with minimal loss of energy to the surrounding environment; ability to ultrasonically alter physical properties (e.g., viscosity) of a fluid via heating or shearing; ability to ultrasonically mix fluid in a tube or pipe to keep multiphase fluids (e.g., oil, gas, water) in a mixed state to, e.g., prevent precipitation of deposits (e.g., asphaltenes) from crude oil; ability to ultrasonically cavitate a fluid to achieve chemical bond cleavage, radical formation, and e.g., enhanced chemical reactivity and enhanced catalysis, or other chemical alterations; ability to ultrasonically repel pipe fouling deposits, such as asphaltenes in the crude oil industry and other deposits that do not respond positively to heat from depositing on the I.D. of production piping; and ability to separate emulsions into separate phases at low temperatures.

The nature and design of the present invention is adaptable to an array of cylindrical containers (e.g., for static fluid processing), and to production piping (e.g., for dynamic fluid processing). For example, available piezoelectric materials allow dimensions of ultrasound devices described herein to be customized to fit cylindrical containers or production piping, e.g., crude oil production piping. In addition, additional devices and components can be used in conjunction with the piezoelectric materials to expand and extend capabilities. For example, transducers could be coupled to conduits and piping with coupling devices such as support clamps that: 1) increase coupling efficiency, 2) change resonance frequency by increasing a desired dimension (e.g., thickness) of the piezoelectric devices; 3) further assist in directing ultrasonic energy into a material or fluid within a pipe or conduit, 3) provide protective cover for coupled transducers, increasing ruggedness. It is further envisioned that external cooling and/or temperature control systems could be used to maintain desired and/or optimum operating conditions of the piezoelectric devices. And, as described herein, the present invention can be utilized in a variety of applications, e.g., performing physical alterations such as heating and mixing for flow assurance; and performing chemical alterations, e.g, for enhanced chemical reactivity and catalysis. For example, the upstream crude oil industry (production) suffers from increased extraction efforts due to highly viscous crude oil and from pipe fouling due to organic (e.g. paraffins) and inorganic wax deposition on the insides of production piping. In crude oil production operations, crude oil is often left in the ground because it cannot be extracted profitably. Highly viscous crude oils require increased energy to extract. And, deposition of pipe-fouling compounds in crude oil pumping equipment is an enormously expensive problem for nearly all oil producers around the world. In the field, production tubing is often plugged by, e.g., paraffin wax that deposits on the walls of the tubing and surface flow equipment. Accumulation of these deposits leads to a significant fall in oil production rates in affected wells. Deposition of paraffin wax occurs when temperature and pressure in the piping move below the cloud point of the oil. Cloud point fluctuations cause paraffin wax crystals to form in the oil and collect within the piping and also increase viscosity of the oil, further choking off flow-lines. Further compounding this problem is the cooling that occurs as oil moves upward through the ground because of lower surface temperatures, ultimately resulting in increased wax precipitation and increased oil viscosity.

During laboratory trials, it was observed that crude oil in the central axial zone of a pipe experiences a temperature increase long before the pipe or PZT rings do. Eventually, the pipe and rings warm and the processing system as a whole begins to thermally equilibrate. This rapid, efficient heating of the oil provided by the present invention has the ability to reduce crude oil viscosity for easier extraction. It is reasonable to believe that the ultrasonic vibrational energy provided by the invention may also mitigate accumulation of deposits on the inner walls and diameters of pipes (e.g., within the volume or string of the piezoelectric transducers) that lead to pipe fouling and increased maintenance. Through mixing, the technology has the ability to keep water and oil in a desired colloidal suspension that is believed to prevent other compounds, such as asphaltenes, from forming pipe-fouling deposits that do not respond positively to heat. In sum, ultrasonic volumetric processing technology of the present invention can simultaneously address these and other issues thereby increasing crude oil production, decreasing maintenance costs, and increasing overall profitability of industrial oil wells.

From the foregoing it will be clear that the invention can be applied to various industrial applications including, but not limited to, e.g., upstream and downstream petroleum processing, chemical manufacturing, pharmaceuticals and pharmaceutical manufacturing, biomass-to-fuel conversion, and waste water treatment. The ultrasonic technology described herein has demonstrated the ability to physically and chemically alter fluids with cavitating and non-cavitating ultrasound. It can also enhance chemical reactivity, e.g., in combination with introduction of catalysts and nanocatalysts. While various preferred embodiments of the invention have been shown and described, it is to be distinctly understood that this invention is not limited thereto but may be variously embodied to practice within the scope of the following claims. And, it will be understood and readily apparent that various changes may be made without departing from the spirit and scope of the invention as defined by the following claims.

We claim:

1. A device comprising:
a container having a circumvolving outer wall adapted to allow passage of a preselected quantity of a material within a passageway defined by said container, said passageway substantially free from obstruction;
at least one ultrasonic energy transmitting device connected to and substantially circumvolving said outer wall outside of said container; and
whereby said at least one ultrasonic energy transmitting device transmits ultrasonic energy having preselected characteristics to said material within said container.

2. The device of claim 1, wherein said container is a conduit.

3. The device of claim 1, wherein said ultrasonic energy transmitting device is a generally cylindrically shaped piezoelectric transducer that transmits energy symmetrically through the outer wall of said container to a central location within said container.

4. The device of claim 1, wherein said ultrasonic energy transmitting device is configured to provide ultrasonic energy in at least one modality.

5. The device of claim 1, wherein said ultrasonic energy transmitting device is configured to provide ultrasonic energy in at least two different modalities.

6. The device of claim 1, wherein said ultrasonic energy transmitting device is configured to provide ultrasonic energy in at least three different modalities.

7. The device of claim 1, wherein said preselected characteristics include an impedance that matches the acoustic impedance of the outer wall of said container.

8. The device of claim 1, further comprising a chemical agent within said container wherein said chemical agent interacts with said ultrasonic energy to produce a desired result within said material.

9. The device of claim 1, further comprising at least one other ultrasonic energy transmitting device connected to and substantially circumvolving said outer wall outside of said container, said at least one other ultrasonic transmitting device positioned along said conduit at a distance about equal to one half of the wavelength of the energy transmitted by said energy transmitting device.

10. The device of claim 8, wherein at least two of said ultrasonic energy transmitting devices provide ultrasonic energy in a different modality to said material within said container.

11. The device of claim 9, further comprising a bank of at least three ultrasonic energy transmitting devices connected to and substantially circumvolving said outer wall of said container outside of said container, each of said at least three ultrasonic energy transmitting devices spaced along said container at a distance of at least about one half of the wavelength of the energy transmitted by said energy transmitting devices.

12. The device of claim 1, further comprising at least one other ultrasonic energy transmitting device connected to and substantially circumvolving said outer wall outside of said container, said at least one other ultrasonic transmitting device spaced along said container at a distance other than about one half of the wavelength of the energy transmitted by said energy transmitting device along said container.

13. The device of claim 1, wherein said at least one ultrasonic energy transmitting device includes said container as a component thereof.

14. A method for treating a material within a walled container substantially free from internal obstructions said method characterized by:
transmitting ultrasonic energy having preselected characteristics to said material from an ultrasonic energy transmission device that is connected to and substantially circumvolves an outer portion of said walled container.

15. The method of claim 14, wherein said ultrasonic energy transmission device comprises a generally cylindrically shaped ring adapted to surround and connect to said outer portion of said walled container.

16. The method of claim 14, wherein said ultrasonic energy transmission device comprises a plurality of ultrasonic energy transmission devices connected to said outer portion of said walled container so as to substantially circumvolve said walled container.

17. The method of claim 16, wherein said plurality of ultrasonic energy transmission devices are bonded to said walled container in the same plane.

18. The method of claim 16, wherein said plurality of ultrasonic energy transmission devices are bonded to said walled container in different planes.

19. The method of claim 16, wherein said plurality of ultrasonic energy transmission devices comprise at least two generally semicircular shaped ultrasonic energy transducers that substantially surround said walled container.

20. The method of claim 16, wherein said plurality of ultrasonic energy transmission devices comprise at least three ultrasonic energy transducers that substantially surround said walled container.

21. A method for heating materials within a walled container substantially free from internal obstructions said method characterized by:
transmitting ultrasonic energy having preselected characteristics to said material from an ultrasonic energy transmission device that is connected to and substantially circumvolves an outer portion of said walled container.

22. A method for performing cavitation of a material within a walled container substantially free from internal obstructions said method characterized by::
transmitting ultrasonic energy having preselected characteristics to said material from an ultrasonic energy transmission device that is connected to and substantially circumvolves an outer portion of said walled container.

23. A method for refining materials in a walled container substantially free from internal obstructions said method characterized by:
transmitting ultrasonic energy transmitting ultrasonic energy having preselected characteristics to said material from an ultrasonic energy transmission device that is connected to and substantially circumvolves an outer portion of said walled container; and
whereby transmission of ultrasonic energy to said material is accomplished without interference with the flow of materials through the walled conduit.

24. A method for preventing the accumulation of deposits within a conduit characterized by:
transmitting ultrasonic energy transmitting ultrasonic energy having preselected characteristics to said material from an ultrasonic energy transmission device that is connected to and substantially circumvolves an outer portion of said conduit; and
whereby transmission of ultrasonic energy to said material is accomplished without the imposition of a physical item within the walled conduit.

25. A method for reducing the viscosity of materials flowing through a walled conduit characterized by:
transmitting ultrasonic energy having preselected characteristics to said material from an ultrasonic energy transmission device that is connected to and substantially circumvolves an outer portion of said conduit; and
whereby transmission of ultrasonic energy to said material is accomplished without the imposition of a physical item within said walled conduit.
